(19) United States
(12) Patent Application Publication
(10) Pub. No.: US 2016/0043533 A1
(43) Pub. Date: Feb. 11, 2016

(54) Process For Providing A Contamination-Reducing Component To An Electrical Apparatus

(71) Applicant: ABB Technology AG, Zurich (CH)

(72) Inventors: Denis Tehlar, Zurich (CH); Navid Mahdizadeh, Baden (CH); Patrick Stoller, Illnau (CH); Thomas Alfred Paul, Wadenswil (CH)

(21) Appl. No.: 14/920,564

(22) Filed: Oct. 22, 2015

Related U.S. Application Data
(63) Continuation of application No. PCT/EP2014/057803, filed on Apr. 16, 2014.

(30) Foreign Application Priority Data
Apr. 22, 2013 (EP) PCT/EP2013/058316

(51) Int. Cl. H02B 13/055; G01N 7/04; G01N 33/00; H02B 13/035; H02B 3/00

(52) U.S. Cl. CPC H02B 13/055; H02B 13/035; H02B 3/00; G01N 33/004; G01N 7/04

(57) ABSTRACT

A process for providing a contamination-reducing component to an electrical apparatus, the electrical apparatus including a housing enclosing an insulating space and an electrical component arranged in the insulating space, the insulating space including an insulation medium which includes or consists of carbon dioxide. The process includes the steps of presaturating the contamination-reducing component with carbon dioxide before placing it inside the electrical apparatus.

### FIELD OF THE INVENTION

The present invention relates to a process for providing a contamination-reducing component in an electrical apparatus, according to the invention.

### BACKGROUND OF THE INVENTION

Further, the present invention relates to an electrical apparatus as well as to a process for determining a change in the sorption capacity, particularly adsorption capacity, of a contamination-reducing component in an electrical apparatus. Dielectric insulation media in liquid or gaseous state are conventionally applied for the insulation of an electrical component in a wide variety of electrical apparatuses, such as for example switchgears, gas-insulated substations (GIS), gas-insulated lines (GIL), transformers, or other.

In medium or high voltage metal-encapsulated switchgears, for example, the electrical component is arranged in a gas-tight housing, which defines an insulating space, said insulating space comprising an insulation medium and separating the housing from the electrical component without letting electrical current to pass through the insulating space.

For interrupting the current in high voltage switchgear, the insulating medium further functions as an arc extinction medium.

In this regard, an insulation medium comprising or consisting of carbon dioxide (CO₂) has been shown to be highly advantageous, due its high arc extinction capability. Further, carbon dioxide has a fairly low impact on the environment. Considering environmental friendliness, it is, thus, a suitable substitute for SF₆ (sulphur hexafluoride), which has commonly been used as a dielectric insulation medium, but which is known to have a high Global Warming Potential (GWP).

In the past, adsorbers have been used as a contamination-reducing component.

However, in connection with an insulation medium comprising carbon dioxide, the known adsorbers have the disadvantage that not only moisture and—as the case may be—decomposition products are adsorbed, but also carbon dioxide and—in case of a carbon dioxide comprising mixture—potentially other constituents of the insulation medium. When placing the adsorber into the insulating space of the electrical apparatus, an initial adsorption of constituents of the insulation medium thus takes place, which has an unwanted effect on the total pressure and on the composition of the insulation medium, affecting both the insulation and arc-extinguishing performance of the medium.

In order to compensate for this initial adsorption, there are in general two possible strategies:

According to a first strategy, more of the insulation medium than required during operation is filled at the initial filling of the insulating space before adding the adsorber, in order to compensate the initially adsorbed amount of insulation medium. This requires, however, detailed knowledge of the size of the insulating space as well as of the amount of the adsorber and its adsorption capability.

According to a second strategy, the gas compartment is refilled after initial adsorption has taken place. This has the disadvantage of a relatively long waiting period, namely the period between the placing of the adsorber into the insulating space and the time until when initial adsorption is complete, before refilling can be performed.

When a gas mixture is used as insulation medium, both of the above described possible strategies require knowledge of the adsorption behaviour of the adsorber towards the constituents of the mixture in order to attain the desired composition of the insulation medium.

Often, carbon dioxide has higher adsorption energy towards the adsorber than the other constituents of the gas mixture. In this case, the relative amount of carbon dioxide in proximity to the adsorber is reduced immediately after the placing of the adsorber into the insulating space. Depending on the diffusion or convection processes of the gas, it can take time for the mixture in the insulating space to become uniform again.

To minimize these effects, the amount of adsorber placed in the insulating space can be reduced to a minimum. In particular in an electrical apparatus comprising multiple compartments, i.e. multiple separate insulating spaces, the determination of the required minimum for each separate insulating space is not only time-consuming. It also bears the risk that during assembly of the electrical apparatus the respective adsorbers are not correctly attributed to their compartments and are thus placed in the wrong insulating space.

With regard to a CO₂ gas circuit breaker, it has been suggested in EP-A-2 445 068 to use a zeolite to which CO₂ molecules are adsorbed in advance before use of the gas circuit breaker. This suggestion is based on the idea that when the CO₂ molecules are adsorbed to the zeolite in advance, the CO molecules which are generated as a result of arcing (and which would otherwise lead to deterioration of the insulation performance and the arc-extinguishing performance) are adsorbed to the zeolite, and the CO₂ molecules that have been adsorbed to the zeolite are released. Ultimately, the increase in the amount of CO as well as the reduction in the amount of CO₂ gas shall thus be suppressed.

Specifically, EP-A-2 445 068 describes a process in which a high-voltage unit and a zeolite case are arranged at predetermined positions inside the closed vessel of an electrical apparatus, and the closed vessel is evacuated. Subsequently, CO₂ gas is enclosed with high pressure in the closed vessel to adsorb CO₂ gas to the zeolite. After that, the predetermined insulation gas is filled into the closed vessel.

The process proposed in EP-A-2 445 068 is, however, relatively laborious, since it requires a large space to be evacuated and then filled with CO₂ for the adsorption to take place, before the predetermined insulation gas is filled in the vessel. This is not only time-consuming, but requires relatively sophisticated CO₂ storage tanks and filling means given the high amount of CO₂ to be filled in the vessel for adsorbing CO₂ to the zeolite.

Considering the shortcomings of the processes of the state of the art, the problem of the present invention is to provide a process for providing a contamination-reducing component to an electrical apparatus in a manner to maintain the insulating performance of the insulation medium contained therein on a high level, said process being efficient, fast and economic.

### SUMMARY OF THE INVENTION

The problem is solved by the subject-matter of the independent claims, and in particular by the process according to the invention. Preferred embodiments of the process of the invention are given in the dependent claims and in claim combinations.

Specifically, the present invention relates to a process for providing a contamination-reducing component to an electrical apparatus, the electrical apparatus comprising a housing enclosing an insulating space and an electrical component arranged in the insulating space, the insulating space comprising an insulation medium which comprises or consists of carbon dioxide. The process comprises the steps of:
a) providing a pre-saturation vessel which is closable in a gas-tight manner and which in its closed state encloses a pre-saturation space, the volume of which being smaller than the volume of the insulating space of the electrical apparatus,
b) placing a contamination-reducing component in the pre-saturation space,
c) filling a pre-saturation gas comprising or consisting of carbon dioxide into the pre-saturation space such as to allow the contamination-reducing component placed in the pre-saturation space to sorb, in particular adsorb, carbon dioxide, and
d) transferring the contamination-reducing component with the sorbed, in particular adsorbed, carbon dioxide to the electrical apparatus such that during operation of the electrical apparatus it comes into contact with the insulation medium.

In the invention, the electrical apparatus is provided with a contamination-reducing component to reduce or eliminate the presence of contaminants, in particular moisture (i.e. water) and/or decomposition products and/or any other component the presence of which is not desired.

In an embodiment, the contamination-reducing component can be a moisture-reducing component. The reduction or elimination of moisture is of particular relevance, since water can—apart from reducing insulation performance—also lead to corrosion of the electrical apparatus, in particular of the housing or the electrical component(s). Further, water can open reaction pathways for the formation of toxic and/or corrosive decomposition products, in particular resulting from partial discharge or arcing in the presence of high moisture content. This is of particular relevance when using an insulation medium which comprises an organofluorine compound, since one decomposition product of the organofluorine compound is hydrogen fluoride (HF), which is highly corrosive and extremely toxic.

The term “housing” as used in the context of the present invention is to be understood broadly as any at least approximately closed system. In particular, the term “housing” can encompass a plurality of chambers interconnected with each other. More particularly in embodiments, “housing” can encompass a chamber, which encloses the insulating space, and a recycling system, the chamber being interconnected with the recycling system through which the insulation medium is removed, processed (e.g. cleaned) and reintroduced into the insulating space. Alternatively or in addition, “housing” can comprise a chamber, which encloses the insulating space, and a pre-treatment room, the chamber being interconnected with the pre-treatment room for pre-treating the insulation medium prior to introduction into the insulating space of the chamber.

Specifically, the “pre-saturation vessel” according to the present invention is separate from the “housing”, i.e. the pre-saturation space is a different enclosed space than the insulating space.

The term “sorption” as used in the context of the present invention is to be interpreted broadly and encompasses any physical or chemical process by which a first substance, i.e. the sorbate, is attached to a second substance, i.e. the sorbent. In particular, “sorption” encompasses any binding, capturing or generally immobilization of the sorbate by any mechanism, such as e.g. by physisorption and/or chemisorption.

According to a specific embodiment of the present invention, the term “sorption” relates to “adsorption”.

In this embodiment, the contamination-reducing component is allowed to adsorb carbon dioxide in step c) and the contamination-reducing component with the adsorbed carbon dioxide is transferred to the electrical apparatus in step d).

The term “adsorption” or “adsorbed” as used in the context of the present invention shall encompass any type of adsorption, such as, e.g., physisorption and/or chemisorption.

According to an embodiment, the steps of the process are consecutive steps.

According to another embodiment, the contamination-reducing component is in step d) transferred into the insulating space of the electrical apparatus. This is not an essential feature of the present invention as the contamination-reducing component, in particular the adsorber, might also be placed elsewhere in the electrical apparatus, e.g. as part of a filter of a recycling system through which the insulation medium is removed from the insulating space, processed (e.g. cleaned) and reintroduced into the insulating space.

Due to the fact that carbon dioxide and, optionally, any other constituent of the pre-saturation gas is pre-sorbed, specifically pre-adsorbed, to the contamination-reducing component, more particularly the molecular sieve, which component or molecular sieve thereby becomes “pre-saturated”, the placing of the contamination-reducing component into the insulating space interferes with the composition of the insulation medium only to a minor degree or not at all. This is explained by the fact that after pre-saturation, generally, most of the sorption sites, specifically the adsorption sites, of the contamination-reducing component are occupied by the constituents of the pre-saturation gas, and particularly by carbon dioxide.

Thus, when placing the contamination-reducing component into the insulating space in the manner according to the present invention, there is no significant change in the total pressure of the insulation medium or no change at all. Also—in case of the insulation medium being a gas mixture—there is no significant change in the gas mixture composition (and, thus, in the ratio of the respective constituents) or no change at all.

As a consequence, the placing of the contamination-reducing component into the insulating space does not interfere with the dielectric performance and—in the respective applications—the switching capabilities of the electrical apparatus.

Although on the one hand carbon dioxide in the insulation medium does not adsorb to the contamination-reducing component (or only to an insignificant degree), water can on the other hand be efficiently removed from the insulation medium due to its higher adsorption capacity than carbon dioxide.

Given the fact that according to the present invention the amount and/or size of the contamination-reducing component does no longer have a substantive effect on the total pressure and—as the case may be—the gas composition of the insulation medium, a contamination-reducing component of relatively large size and/or amount can be used which is able to adsorb large amounts of moisture and/or decomposition products. Thus, a long operating time of the apparatus can be achieved before replacement of the contamination-reducing component becomes necessary.

In addition, in an electrical apparatus comprising multiple compartments, the size and/or amount of the contamination-reducing components to be used for the different insulating spaces can be standardized to the largest insulating space, thus contributing to a simple and safe assembly of the electrical apparatus.

In the context of the present invention, it has surprisingly been found that pre-saturation of the contamination-reducing component in a vessel other than the insulating space of the electrical apparatus can be performed in a simple manner without the risk of release of the adsorbed carbon dioxide during the transfer into the insulating space. This is due to a strong hysteresis between adsorption and release of carbon dioxide. Thus, carbon dioxide can remain adsorbed even in the case that during the transfer according to step d) the contamination-reducing component is exposed to an environment having a number density of carbon dioxide lower than in the pre-saturation space. This is in particular the case, when a low temperature is maintained during the transfer.

According to an embodiment of the present invention, the contamination-reducing component with the carbon dioxide sorbed, specifically adsorbed, is, prior to or during step d), taken out of the pre-saturation space. Particularly, the contamination-reducing component is taken out of the pre-saturation space before being placed into the insulating space of the electrical apparatus.

In this regard, it is particularly preferred that the contamination-reducing component is packaged into a container, in particular a bag, prior to being taken out of the pre-saturation space, said container being moveable with regard to the pre-saturation vessel and the electrical apparatus. Thus, the contamination-reducing component can be stored over a long period of time without losing carbon dioxide adsorbed thereto and thus its desired quality. According to a particularly preferred embodiment, the container is closeable in a gas-tight manner. Thus, a well-defined gas composition and pressure can be provided in the container, which allows to avoid the risk of the contamination-reducing component adsorbing unwanted contaminants and/or releasing carbon dioxide during the transfer.

Although preferred, the packaging into a container is, however, not essential due to the hysteresis between carbon dioxide adsorption and release described above. This holds true especially in the case where the placing of the contamination-reducing component into the insulating space is performed shortly after it has been taken out of the pre-saturation space.

Alternatively to the above embodiment, in which the contamination-reducing component with the carbon dioxide adsorbed is, prior or during step d), taken out of the pre-saturation space, it can—depending on the circumstances—also be preferred to transfer the pre-saturation vessel together with the contamination-reducing component placed in the pre-saturation space to the electrical apparatus and to open the pre-saturation vessel after step d). Thus, the contamination-reducing component is only exposed to the pre-saturation space and the insulating space, the gas composition and pressure in both spaces being well defined. There is thus no risk of the contamination-reducing component adsorbing unwanted contaminants and/or releasing carbon dioxide in this embodiment.

According to an embodiment, the contamination-reducing component is a molecular sieve, more preferably a zeolite, i.e. a micro-porous aluminosilicate mineral that has undergone cation exchange to achieve a desired pore size.

Preferably, the molecular sieve has an average pore size greater than 2 Å, preferably greater than 4 Å, more preferably greater than 5 Å, even more preferably greater than 6 Å, and most preferably greater than 8 Å.

It is a further embodiment that the molecular sieve has a pore size from 3 Å to 13 Å, preferably from 5 Å to 13 Å, more preferably from 6 Å to 13 Å or from 6 Å to 12 Å, even more preferably from 7 Å to 11 Å, most preferably from 9 Å to 11 Å. A respective molecular sieve has been found to have a particularly high adsorption capacity not only for water, but also for e.g. hydrogen fluoride, a potential decomposition product of an insulation medium comprising an organofluorine compound.

Suitable zeolites include e.g. ZEOCHEM® molecular sieve 5A (having a pore size of 5 Å) and ZEOCHEM® molecular sieve 13X (having a pore size of 9 Å).

As mentioned, the term “adsorption” or “adsorbed” encompasses both physisorption and/or chemisorption. Physisorption can, in particular, be determined or be influenced by the relationship between the size of molecules of the insulation medium and the pore size of the molecular sieve. Chemisorption can, in particular, be determined or influenced by chemical interactions between molecules of the insulation medium and the molecular sieve.

The term “adsorption capacity” as used in the context of the present invention refers to the number of molecules adsorbed (in mole) to the mass of the contamination-reducing component, particularly the molecular sieve, more particularly the zeolite (in kg). Likewise, the term “sorption capacity” as used in the context of the present invention refers to the number of molecules sorbed (in mole) to the mass of the contamination-reducing component, particularly the molecular sieve, more particularly the zeolite (in kg).

In embodiments, in order to achieve a good sorption, specifically adsorption, in the pre-saturation space also at relatively moderate filling pressures, the contamination-reducing component is cooled prior to step c) and/or during step c) and/or prior to step d) and/or during step d), preferably to a temperature below 10° C., more preferably below 0° C., most preferably below −20° C.

In other terms, the contamination-reducing component is preferably cooled, prior to step c) and/or during step c) and/or prior to step d) and/or during step d), to a temperature which is at most 5° C. above, in particular equal to or lower than, the minimum operating temperature of the electrical apparatus which is to be provided with the contamination-reducing component.

In general, the number density of carbon dioxide in the pre-saturation space is higher than the number density of carbon dioxide in air at atmospheric pressure.

Particularly, the number density of carbon dioxide in the pre-saturation space is at least approximately equal to the maximum expected number density of carbon dioxide in the insulating space of the electrical apparatus. More particularly, the partial pressure of carbon dioxide in the pre-saturation space at room temperature is higher than 1 bar, preferably higher than 3 bar, more preferably higher than 5 bar, and most preferably higher than 7 bar. Thus, no carbon dioxide will be adsorbed after transferring the contamination-reducing component to the electrical apparatus (because the adsorption capacity increases with the partial pressure and is therefore higher in the higher-pressure environment of the pre-saturation space than in the lower-pressure environment of the insulating space), and no change in the total pressure and in the composition of the insulation medium will occur.

According to a further preferred embodiment, the volume of the pre-saturation space is slightly greater than the volume of the molecular sieve. Thus, an optimum in adsorption can be achieved in the pre-saturation space, while keeping the required time and cost for filling it up to the required pressure as short and as low as possible.

According to a still further preferred embodiment, the insulation medium and the pre-saturation gas have at least approximately the same composition. Thus, after transferring the contamination-reducing component from the pre-saturation space into the insulating space, no adsorption of constituents having a higher adsorption capacity than an already adsorbed constituent (and, thus, removal of the latter) will occur. If a gas mixture is used as insulation medium, a change in the composition of the latter is prevented in this embodiment.

As mentioned previously, the insulation medium can consist or essentially consist of carbon dioxide. In this embodiment, carbon dioxide is thus the sole component of the insulation medium.

Alternatively, the insulation medium can comprise carbon dioxide apart from other constituents, and, thus, form a gas mixture, which is an often preferred embodiment. It is particularly preferred that the insulation medium comprises air or at least one air component, in particular oxygen and/or nitrogen, apart from carbon dioxide.

According to an embodiment, the insulation medium is a gas mixture comprising carbon dioxide and oxygen. According to a particularly preferred embodiment, the ratio of the amount of carbon dioxide to the amount of oxygen thereby can range from 50:50 to 100:1.

In particular in view of interrupting the current in a high voltage switchgear, it is a further embodiment that the ratio of the amount of carbon dioxide to the amount of oxygen ranges from 80:20 to 95:5, more preferably from 85:15 to 92:8, even more preferably from 87:13 to less than 90:10, and in particular is about 89:11. In this regard, it has been found on the one hand that oxygen being present in a molar fraction of at least 5% allows soot formation to be prevented even after repeated current interruption events with high current arcing. On the other hand, oxygen being present in a molar fraction of at most 20%, more particularly of at most 15%, reduces the risk of degradation of the material of the electrical apparatus by oxidation.

As mentioned previously, the advantageous effects of a contamination-reducing component are particularly pronounced in embodiments in which the insulation medium comprises an organofluorine compound, since thereby the generation of harmful decomposition products, such as hydrogen fluoride, which in the absence of a contamination-reducing component might occur, can efficiently be avoided. These embodiments are thus advantageous in the context of the present invention.

Specifically, the organofluorine compound is selected from the group consisting of fluoroethers, in particular hydrofluoro-monoethers, fluoroketones, in particular perfluoroketones, and fluoroolefins, in particular hydrofluoroolefins, and mixtures thereof.

These classes of compounds have been found to have very high insulation capabilities, in particular a high dielectric strength (or breakdown field strength), and at the same time a low GWP and low toxicity.

Due to the pre-saturation of the contamination-reducing component, i.e. the sorbing of carbon dioxide in the pre-saturation space, the sorption of organofluorine compounds, particularly of a fluoroketone containing from 4 to 12 carbon atoms, specifically exactly 5 carbon atoms, can be efficiently avoided by the process of the present invention. There is thus no loss in the partial pressure of the organofluorine compound due to the transfer of the contamination-reducing component to the electrical apparatus according to step d).

The invention encompasses both embodiments in which the dielectric insulation gas comprises either one of a fluoro-ether, in particular a hydrofluoromonoether, a fluoroketone and a fluoroolefin, in particular a hydrofluoroolefin, as well as embodiments in which it comprises a mixture of at least two of these compounds.

The term “fluoroether” as used in the context of the present invention encompasses both fluoropolyethers (e.g. galden) and fluoromonoethers and encompasses both perfluoroethers, i.e. fully fluorinated ethers, and hydrofluoroethers, i.e. ethers that are only partially fluorinated. The term “fluoroether” further encompasses saturated compounds as well as unsaturated compounds, i.e. compounds including double and/or triple bonds between carbon atoms. The at least partially fluorinated alkyl chains attached to the oxygen atom of the fluoroether can, independently of each other, be linear or branched.

The term “fluoroether” further encompasses both non-cyclic and cyclic ethers. Thus, the two alkyl chains attached to the oxygen atom can optionally form a ring. In particular, the term encompasses fluorooxiranes. In a specific embodiment, the organofluorine compound according to the present invention is a perfluorooxirane or a hydrofluorooxirane, more specifically a perfluorooxirane or hydrofluorooxirane comprising from three to fifteen carbon atoms.

According to other embodiments, the dielectric insulation gas comprises a hydrofluoromonoether containing at least three carbon atoms. Apart from their high dielectric strength, these hydrofluoromonoethers are chemically and thermally stable up to temperatures above 140° C. They are further non-toxic or have a low toxicity level. In addition, they are non-corrosive and non-explosive.

The term “hydrofluoromonoether” as used herein refers to a compound having one and only one ether group, said ether group linking two alkyl groups, which can be, independently from each other, linear or branched, and which can optionally form a ring. The compound is thus in clear contrast to the compounds disclosed in e.g. U.S. Pat. No. 7,128,133, which relates to the use of compounds containing two ether groups, i.e. hydrofluorodiethers, in heat-transfer fluids.

The term “hydrofluoromonoether” as used herein is further to be understood such that the monoether is partially hydrogenated and partially fluorinated. It is further to be understood such that it may comprise a mixture of differently structured hydrofluoromonoethers. The term “structurally different” shall broadly encompass any difference in sum formula or structural formula of the hydrofluoromonoether.

As mentioned above, hydrofluoromonoethers containing at least three carbon atoms have been found to have a relatively high dielectric strength. Specifically, the ratio of the dielectric strength of the hydrofluoromonoethers according to the present invention to the dielectric strength of SF₆ is greater than about 0.4.

As also mentioned, the GWP of the hydrofluoromonoethers is low. Preferably, the GWP is less than 1,000 over 100 years, more specifically less than 700 over 100 years. The hydrofluoromonoethers mentioned herein have a relatively low atmospheric lifetime and in addition are devoid of halogen atoms that play a role in the ozone destruction catalytic cycle, namely Cl, Br or I. Their Ozone Depletion Potential (ODP) is zero, which is very favourable from an environmental perspective.

The preference for a hydrofluoromonoether containing at least three carbon atoms and thus having a relatively high boiling point of more than −20° C. is based on the finding that a higher boiling point of the hydrofluoromonoether generally goes along with a higher dielectric strength.

According to other embodiments, the hydrofluoromonoether contains exactly three or four or five or six carbon atoms, in particular exactly three or four carbon atoms, most preferably exactly three carbon atoms.

More particularly, the hydrofluoromonoether is thus at least one compound selected from the group consisting of the compounds defined by the following structural formulae in which a part of the hydrogen atoms is each substituted by a fluorine atom:

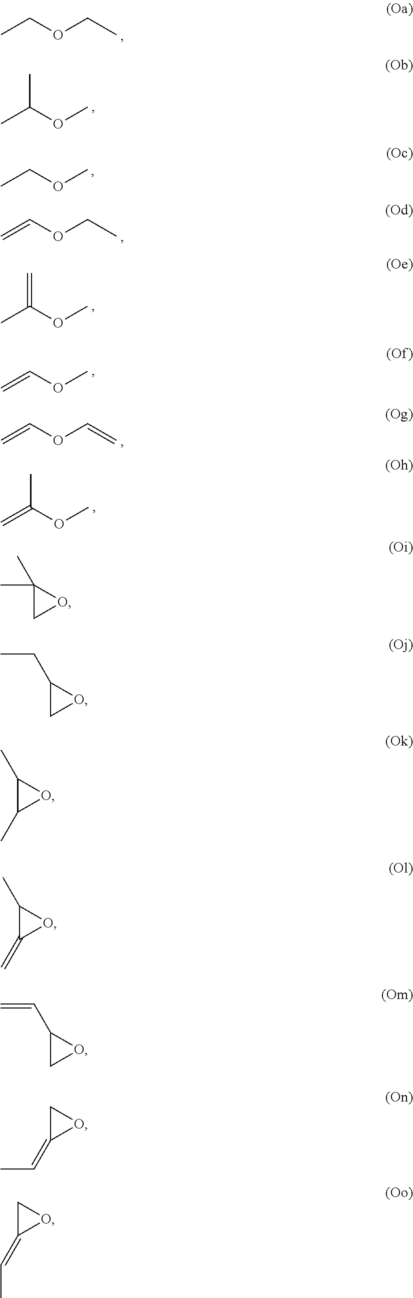

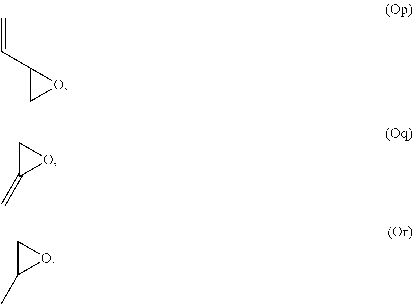

By using a hydrofluoromonoether containing three or four carbon atoms, no liquefaction occurs under typical operational conditions of the apparatus. Thus, a dielectric insulation medium, every component of which is in the gaseous state at operational conditions of the apparatus, can be achieved.

Considering flammability of the compounds, it is further advantageous that the ratio of the number of fluorine atoms to the total number of fluorine and hydrogen atoms, here briefly called “F-rate”, of the hydrofluoromonoether is at least 5:8. It has been found that compounds falling within this definition are generally non-flammable and thus result in an insulation medium complying with highest safety requirements. Thus, safety requirements of the electrical insulator and the method of its production can readily be fulfilled by using a corresponding hydrofluoromonoether.

According to other embodiments, the ratio of the number of fluorine atoms to the number of carbon atoms, here briefly called “F/C-ratio”, ranges from 1.5:1 to 2:1. Such compounds generally have a GWP of less than 1,000 over 100 years and are thus very environment-friendly. It is particularly preferred that the hydrofluoromonoether has a GWP of less than 700 over 100 years.

According to other embodiments of the present invention, the hydrofluoromonoether has the general structure (O)

CₐH_{b}F_{c}—O—C_{d}HₑF_{f} (O)

wherein a and d independently are an integer from 1 to 3 with a+d=3 or 4 or 5 or 6, in particular 3 or 4, b and c independently are an integer from 0 to 11, in particular 0 to 7, with b+c=2a+1, and e and f independently are an integer from 0 to 11, in particular 0 to 7, with e+f=2d+1, with further at least one of b and e being 1 or greater and at least one of c and f being 1 or greater.

It is thereby a preferred embodiment that in the general structure or formula (O) of the hydrofluoromonoether:

a is 1, b and c independently are an integer ranging from 0 to 3 with b+c=3, d=2, e and f independently are an integer ranging from 0 to 5 with e+f=5, with further at least one of b and e being 1 or greater and at least one of c and f being 1 or greater.

According to a more particular embodiment, exactly one of c and f in the general structure (O) is 0. The corresponding grouping of fluorines on one side of the ether linkage, with the other side remaining unsubstituted, is called “segregation”. Segregation has been found to reduce the boiling point compared to unsegregated compounds of the same chain length. This feature is thus of particular interest, because compounds with longer chain lengths allowing for higher dielectric strength can be used without risk of liquefaction under operational conditions.

Most preferably, the hydrofluoromonoether is selected from the group consisting of pentafluoro-ethyl-methyl ether (CH₃—O—CF₂CF₃) and 2,2,2-trifluoroethyl-trifluoromethyl ether (CF₃—O—CH₂CF₃).

Pentafluoro-ethyl-methyl ether has a boiling point of +5.25° C. and a GWP of 697 over 100 years, the F-rate being 0.625, while 2,2,2-trifluoroethyl-trifluoromethyl ether has a boiling point of +11° C. and a GWP of 487 over 100 years, the F-rate being 0.75. They both have an ODP of 0 and are thus environmentally fully acceptable.

In addition, pentafluoro-ethyl-methyl ether has been found to be thermally stable at a temperature of 175° C. for 30 days and therefore to be fully suitable for the operational conditions given in the apparatus. Since thermal stability studies of hydrofluoromonoethers of higher molecular weight have shown that ethers containing fully hydrogenated methyl or ethyl groups have a lower thermal stability compared to those having partially hydrogenated groups, it can be assumed that the thermal stability of 2,2,2-trifluoroethyl-trifluoromethyl ether is even higher.

Hydrofluoromonoethers in general, and pentafluoro-ethyl-methyl ether as well as 2,2,2-trifluoroethyl-trifluoromethyl ether in particular, display a low risk of human toxicity. This can be concluded from the available results of mammalian HFC (hydrofluorocarbon) tests. Also, information available on commercial hydrofluoromonoethers do not give any evidence of carcinogenicity, mutagenicity, reproductive/developmental effects and other chronic effects of the compounds of the present application.

Based on the data available for commercial hydrofluoro ethers of higher molecular weight, it can be concluded that the hydrofluoromonoethers, and in particular pentafluoro-ethyl-methyl ether as well as 2,2,2-trifluoroethyl-trifluoromethyl ether, have a lethal concentration LC 50 of higher than 10,000 ppm, rendering them suitable also from a toxicological point of view.

The hydrofluoromonoethers mentioned have a higher dielectric strength than air. In particular, pentafluoro-ethyl-methyl ether at 1 bar has a dielectric strength about 2.4 times higher than that of air at 1 bar.

Given its boiling point, which is preferably below 55° C., more preferably below 40° C., in particular below 30° C., the hydro-fluoromonoethers mentioned, particularly pentafluoro-ethyl-methyl ether and 2,2,2-trifluoroethyl-trifluoromethyl ether, respectively, are normally in the gaseous state at operational conditions. Thus, a dielectric insulation medium in which every component is in the gaseous state at operational conditions of the apparatus can be achieved, which is advantageous.

Alternatively or additionally to the hydrofluoromonoethers mentioned above, the dielectric insulation gas comprises a fluoroketone containing from four to twelve carbon atoms.

The term “fluoroketone” as used in this application shall be interpreted broadly and shall encompass both perfluoroketones and hydrofluoroketones, and shall further encompass both saturated compounds and unsaturated compounds, i.e. compounds including double and/or triple bonds between carbon atoms. The at least partially fluorinated alkyl chain of the fluoro-ketones can be linear or branched, or can form a ring, which optionally is substituted by one or more alkyl groups. In exemplary embodiments, the fluoroketone is a perfluoroketone. In further exemplary embodiment, the fluoroketone has a branched alkyl chain, in particular an at least partially fluorinated alkyl chain. In still further exemplary embodiments, the fluoroketone is a fully saturated compound.

According to another aspect, the present invention also relates to a dielectric insulation medium comprising a fluoroketone having from 4 to 12 carbon atoms, the at least partially fluorinated alkyl chain of the fluoroketone forming a ring, which is optionally substituted by one or more alkyl groups. Furthermore, such dielectric insulation medium can comprise a background gas, in particular selected from the group consisting of: air, air component, nitrogen, oxygen, nitrogen oxides, carbon dioxide, and mixtures thereof. Furthermore, the invention relates to an electrical apparatus comprising such a dielectric insulation medium.

Compared to fluoroketones having a greater chain length with more than six carbon atoms, fluoroketones containing five or six carbon atoms have the advantage of a relatively low boiling point. Thus, problems which might go along with liquefaction can be avoided, even when the apparatus is used at low temperatures.

According to embodiments, the fluoroketone is at least one compound selected from the group consisting of the compounds defined by the following structural formulae in which at least one hydrogen atom is substituted with a fluorine atom:

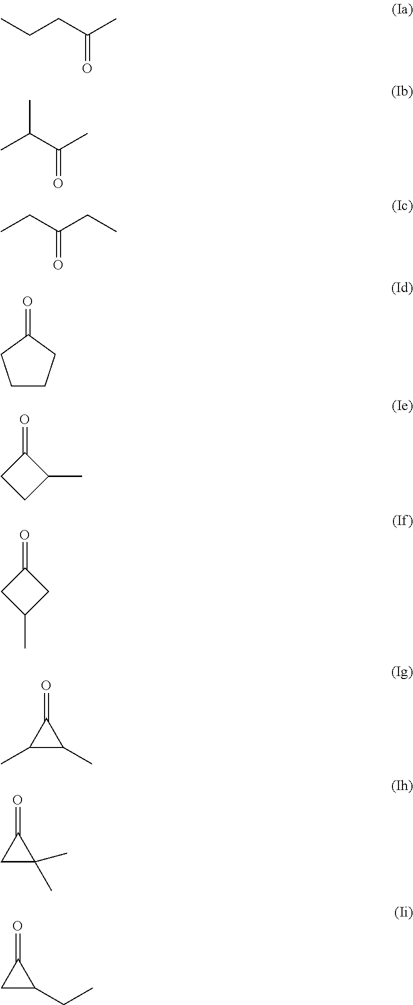

According to another aspect, the present invention relates to a dielectric insulation medium comprising a fluoroketone having exactly 5 carbon atoms and having a structural formula according to (Ia) to (Ii). Furthermore, such dielectric insulation medium can comprise a background gas, in particular selected from the group consisting of: air, air component, nitrogen, oxygen, nitrogen oxides, carbon dioxide, and mixtures thereof. Furthermore, an electrical apparatus comprising such a dielectric insulation medium is disclosed.

Fluoroketones containing five or more carbon atoms are further advantageous, because they are generally non-toxic with outstanding margins for human safety. This is in contrast to fluoroketones having less than four carbon atoms, such as hexafluoroacetone (or hexafluoropropanone), which are toxic and very reactive. In particular, fluoroketones containing exactly five carbon atoms, herein briefly named fluoroketones a), and fluoroketones containing exactly six carbon atoms are thermally stable up to 500° C.

In embodiments of this invention, the fluoroketones, in particular fluoroketones a), having a branched alkyl chain are preferred, because their boiling points are lower than the boiling points of the corresponding compounds (i.e. compounds with same molecular formula) having a straight alkyl chain.

According to embodiments, the fluoroketone a) is a perfluoroketone, in particular has the molecular formula C₅F₁₀O, i.e. is fully saturated without double or triple bonds between carbon atoms. The fluoroketone a) may more preferably be selected from the group consisting of 1,1,1,3,4,4,4-heptafluoro-3-(trifluoromethyl)butan-2-one (also named decafluoro-2-methylbutan-3-one), 1,1,1,3,3,4,4,5,5,5-deca-fluoropentan-2-one, 1,1,1,2,2,4,4,5,5,5-decafluoropentan-3-one and octafluorocylcopentanone, and most preferably is 1,1,1,3,4,4,4-heptafluoro-3-(trifluoromethyl)butan-2-one. 1,1,1,3,4,4,4-heptafluoro-3-(trifluoromethyl)butan-2-one can be represented by the following structural formula (I):

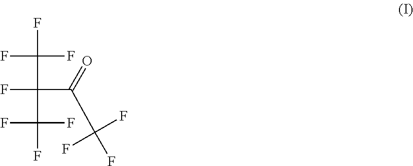 [SMILES: O=C(C(F)(F)F)C(F)(C(F)(F)F)C(F)(F)F]

1,1,1,3,4,4,4-heptafluoro-3-(trifluoromethyl)butan-2-one, here briefly called “C5-ketone”, with molecular formula CF₃C(O)CF(CF₃)₂ or C₅F₁₀O, has been found to be particularly preferred for high and medium voltage insulation applications, because it has the advantages of high dielectric insulation performance, in particular in mixtures with a dielectric carrier gas, has very low GWP and has a low boiling point. It has an ODP of 0 and is practically non-toxic.

According to embodiments, even higher insulation capabilities can be achieved by combining the mixture of different fluoroketone components. In embodiments, a fluoroketone containing exactly five carbon atoms, as described above and here briefly called fluoroketone a), and a fluoroketone containing exactly six carbon atoms or exactly seven carbon atoms, here briefly named fluoroketone c), can favourably be part of the dielectric insulation at the same time. Thus, an insulation medium can be achieved having more than one fluoroketone, each contributing by itself to the dielectric strength of the insulation medium.

In embodiments, the further fluoroketone c) is at least one compound selected from the group consisting of the compounds defined by the following structural formulae in which at least one hydrogen atom is substituted with a fluorine atom:

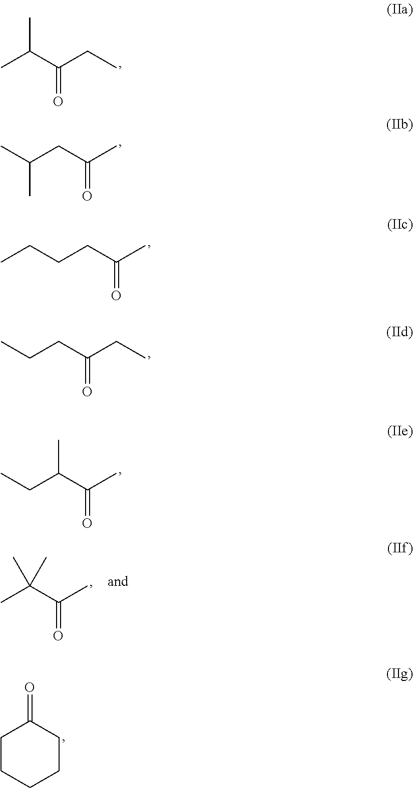

as well as any fluoroketone having exactly 6 carbon atoms, in which the at least partially fluorinated alkyl chain of the fluoroketone forms a ring, which is substituted by one or more alkyl groups (IIh);
and/or is at least one compound selected from the group consisting of the compounds defined by the following structural formulae in which at least one hydrogen atom is substituted with a fluorine atom:

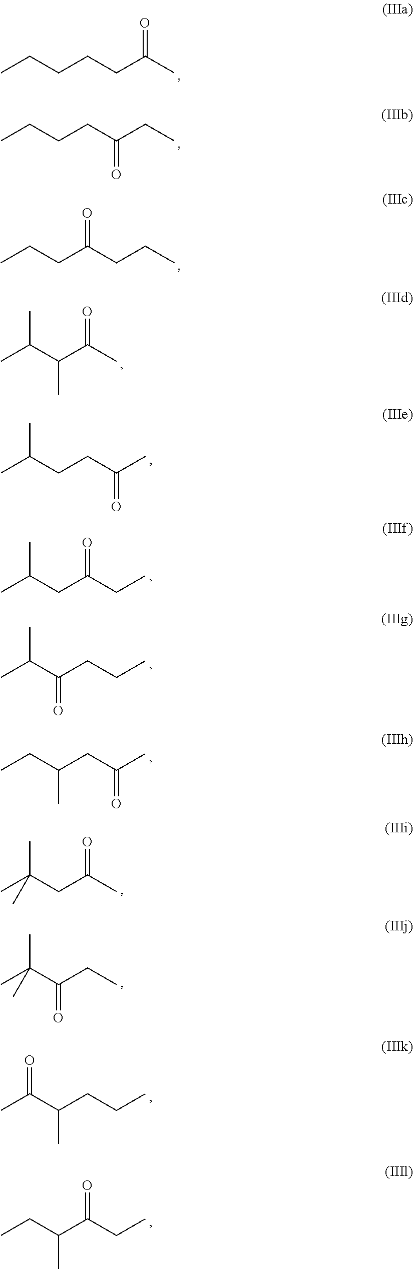

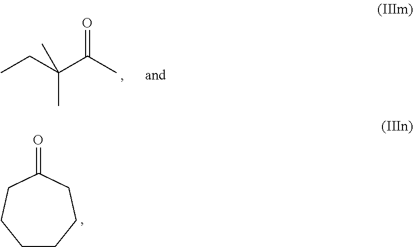

for example dodecafluoro-cycloheptanone, as well as any fluoroketone having exactly 7 carbon atoms, in which the at least partially fluorinated alkyl chain of the fluoroketone forms a ring, which is substituted by one or more alkyl groups (IIIo).

According to another aspect, the present invention relates to a dielectric insulation medium comprising a fluoroketone having exactly 6 carbon atoms, in which the at least partially fluorinated alkyl chain of the fluoroketone forms a ring, optionally substituted by one or more alkyl groups. Furthermore, such dielectric insulation medium can comprise a background gas, in particular selected from the group consisting of: air, air component, nitrogen, oxygen, nitrogen oxides, carbon dioxide, and mixtures thereof. Furthermore, an electrical apparatus comprising such a dielectric insulation medium is disclosed.

According to still another aspect, the present invention relates to a dielectric insulation medium comprising a fluoroketone having exactly 7 carbon atoms, in which the at least partially fluorinated alkyl chain of the fluoroketone forms a ring, optionally substituted by one or more alkyl groups. Furthermore, such dielectric insulation medium can comprise a background gas, in particular selected from the group consisting of: air, air component, nitrogen, oxygen, nitrogen oxides, carbon dioxide, and mixtures thereof. Furthermore, an electrical apparatus comprising such a dielectric insulation medium is disclosed.

The present invention encompasses each compound or each combination of compounds selected from the group consisting of the compounds according to structural formulae (Oa) to (Or), (Ia) to (Ii), (IIa) to (IIh), (IIIa) to (IIIo), and mixtures thereof.

Depending on the specific application of the apparatus of the present invention, a fluoroketone containing exactly six carbon atoms (falling under the designation “fluoroketone c)” mentioned above) may be preferred; such a fluoroketone is non-toxic, with outstanding margins for human safety.

In embodiments, fluoroketone c), alike fluoroketone a), is a perfluoroketone, and/or has a branched alkyl chain, in particular an at least partially fluorinated alkyl chain, and/or the fluoroketone c) contains fully saturated compounds. In particular, the fluoroketone c) has the molecular formula C₆F₁₂O, i.e. is fully saturated without double or triple bonds between carbon atoms. More preferably, the fluoroketone c) can be selected from the group consisting of 1,1,1,2,4,4,5,5,5-nonafluoro-2-(trifluoromethyl)pentan-3-one (also named dodecafluoro-2-methylpentan-3-one), 1,1,1,3,3,4,5,5,5-nonafluoro-4-(trifluoromethyl)pentan-2-one (also named dodecafluoro-4-methylpentan-2-one), 1,1,1,3,4,4,5,5,5-nonafluoro-3-(trifluoromethyl)pentan-2-one (also named dodecafluoro-3-methylpentan-2-one), 1,1,1,4,4,4-hexafluoro-3,3-bis-(trifluoromethyl)butan-2-one (also named dodecafluoro-3,3-(dimethyl)butan-2-one), dodecafluorohexan-2-one, dodecafluorohexan-3-one and decafluorocyclohexanone (with sum formula C₆F₁₀O), and particularly is the mentioned 1,1,1,2,4,4,5,5,5-nonafluoro-2-(trifluoromethyl)pentan-3-one.

1,1,1,2,4,4,5,5,5-Nonafluoro-2-(trifluoromethyl)pentan-3-one (also named dodecafluoro-2-methylpentan-3-one) can be represented by the following structural formula (II):

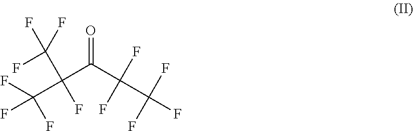 [SMILES: O=C(C(F)(F)C(F)(F)F)C(F)(C(F)(F)F)C(F)(F)F]

1,1,1,2,4,4,5,5,5-Nonafluoro-4-(trifluoromethyl)pentan-3-one (here briefly called “C6-ketone”, with molecular formula C₂F₅C(O)CF(CF₃)₂) has been found to be particularly preferred for high voltage insulation applications because of its high insulating properties and its extremely low GWP. Specifically, its pressure-reduced breakdown field strength is around 240 kV/(cm*bar), which is much higher than the one of air having a much lower dielectric strength (E_{cr}=25 kV/(cm*bar). It has an ozone depletion potential of 0 and is non-toxic (LC50 of about 100,000 ppm). Thus, the environmental impact is much lower than when using SF₆, and at the same time outstanding margins for human safety are achieved.

As mentioned above, the organofluorine compound can also be a fluoroolefin, in particular a hydrofluoroolefin. More particularly, the fluoroolefin or hydrofluorolefin, respectively, contains exactly three carbon atoms.

According to a particularly preferred embodiment, the hydrofluoroolefin is, thus, selected from the group consisting of: 1,1,1,2-tetrafluoropropene (HFO-1234yf), 1,2,3,3-tetrafluoro-2-propene (HFO-1234yc), 1,1,3,3-tetrafluoro-2-propene (HFO-1234zc), 1,1,1,3-tetrafluoro-2-propene (HFO-1234ze), 1,1,2,3-tetrafluoro-2-propene (HFO-1234ye), 1,1,1,2,3-pentafluoropropene (HFO-1225ye), 1,1,2,3,3-pentafluoropropene (HFO-1225yc), 1,1,1,3,3-pentafluoropropene (HFO-1225zc), (Z)1,1,1,3-tetrafluoropropene (HFO-1234zeZ), (Z)1,1,2,3-tetrafluoro-2-propene (HFO-1234yeZ), (E)1,1,1,3-tetrafluoropropene (HFO-1234zeE), (E)1,1,2,3-tetrafluoro-2-propene (HFO-1234yeE), (Z)1,1,1,2,3-pentafluoropropene (HFO-1225yeZ), (E)1,1,1,2,3-pentafluoropropene (HFO-1225yeE) and mixtures thereof.

According to a further aspect, the present invention also relates to an electrical apparatus, in particular obtainable by a process described herein.

In analogy to the above description of the process, the electrical apparatus comprises a housing enclosing an insulating space and an electrical component arranged in the insulating space, said insulating space containing an insulation medium which comprises or consists of carbon dioxide.

According to this aspect of the invention, a molecular sieve having an average pore size in a range from 5 Å to 13 Å is arranged in the insulating space.

In particular, the purpose of the molecular sieve is primarily to reduce or eliminate the presence of contaminants, in particular moisture (i.e. water) and/or decomposition products and/or any other component the presence of which is not desired. Since the reduction or elimination of water is of particularly high relevance, the molecular sieve is preferably a water-reducing component.

Any preferred feature described with regard to the process likewise applies to the electrical apparatus and vice versa.

The molecular sieve is thus preferably a zeolite.

In embodiments, the molecular sieve, specifically the zeolite, has preferably an average pore size greater than 5 Å, more preferably greater than 6 Å, and most preferably greater than 8 Å.

As has been mentioned above and as will be shown in detail below, the molecular sieve, in particular the zeolite, according to the present invention has a very high adsorption capacity towards water and decomposition products, specifically hydrogen fluoride. Preferably, the molecular sieve has an average pore size from 6 Å to 13 Å or from 6 Å to 12 Å, even more preferably from 7 Å to 11 Å, most preferably from 9 Å to 11 Å.

Since the electrical apparatus of the present invention is preferably obtainable by the process described hereinbefore, the molecular sieve is preferably arranged in the pre-saturation space of a pre-saturation vessel as defined above, said pre-saturation vessel being in its open state.

According to a further embodiment of the electrical apparatus, the insulation medium comprises, apart from carbon dioxide, an additional background gas, in particular selected from the group consisting of: air, air component, nitrogen, oxygen, nitrogen oxides, and mixtures thereof.

In embodiments, the ratio of the amount of carbon dioxide to the amount of oxygen ranges from 50:50 to 100:1, preferably from 80:20 to 95:5, more preferably from 85:15 to 92:8, even more preferably from 87:13 to less than 90:10, and most preferably is about 89:11, as has already been described in the context of the process defined above.

In embodiments, the insulation medium further comprises an organofluorine compound, preferably an organofluorine compound selected from the group consisting of: fluoroethers including fluoropolyethers and fluoromonoethers, in particular hydro-fluoromonoethers; fluoroketones, in particular perfluoro-ketones; fluoroolefins, in particular hydrofluoroolefins; and mixtures thereof.

According to an embodiment, the electrical component of the electrical apparatus is a high voltage or medium voltage unit, since in these the task of controlling and delimiting the moisture content is of high importance and the advantages achieved by the present invention are, thus, of particular relevance.

In embodiments, the electrical apparatus can be a switchgear, in particular a gas-insulated switchgear (GIS) or a part and/or component thereof, a busbar, a bushing, a cable, a gas-insulated cable, a cable joint, a gas-insulated line (GIL), a transformer, a current transformer, a voltage transformer, a surge arrester, an earthing switch, a disconnector, a combined disconnector and earthing switch, a load-break switch, a circuit breaker, a convertor building and/or any type of gas-insulated switch.

According to still a further aspect, the present invention further relates to a process for determining a change in the adsorption capacity of a contamination-reducing component in an electrical apparatus.

As mentioned for the embodiments described above, the electrical apparatus comprises a housing enclosing an insulating space and an electrical component arranged in the insulating space. The insulating space comprises an insulation medium which comprises or consists of carbon dioxide.

According to a still further aspect, the present invention also relates to a process for determining and/or monitoring the sorption capacity, in particular adsorption capacity, of a contamination-reducing component in an electrical apparatus, said electrical apparatus comprising a housing enclosing an insulating space and an electrical component arranged in the insulating space, said insulating space comprising an insulation medium which comprises or consists of carbon dioxide.

The process comprises the steps of:
I) providing to the insulating space a contamination-reducing component, in particular a molecular sieve, with at least one sorbate sorbed thereto, said at least one sorbate comprising carbon dioxide,
II) determining an amount of carbon dioxide released from the contamination-reducing component, and
III) determining from the amount determined in step II) the amount of the remaining sorbates in the contamination-reducing component, in particular water and/or decomposition products, and thus the sorption capacity of the contamination-reducing component.

Specifically, the present invention relates to process for determining the sorption capacity of contamination reducing component in an electrical apparatus, said electrical apparatus comprising a housing enclosing an insulating space and an electrical component arranged in the insulating space, said insulating space comprising an insulation medium which comprises or consists of carbon dioxide. The process comprises the steps of:
A) providing to the insulating space a contamination-reducing component, in particular a molecular sieve, with at least one sorbate sorbed (or adsorbate adsorbed) thereto, said at least one sorbate (or adsorbate) comprising carbon dioxide,
B) inducing an at least partial release of sorbate (or adsorbate) from the contamination-reducing component,
C) determining the amount of carbon dioxide released from the contamination-reducing component, and
D) determining from the amount determined in step C) the amount of the remaining sorbates (or adsorbates) in the contamination-reducing component, in particular water and/or decomposition products, and thus the sorption capacity (or adsorption capacity, respectively) of the contamination-reducing component.

In embodiments, the term “adsorbate” as used in the context of the present invention relates to a substance adsorbed to the contamination-reducing agent. In addition to carbon dioxide, at least one further substance or adsorbate can be adsorbed. In this context, the expression “at least one adsorbate” is equivalent to the expression “at least one kind of adsorbate”.

The release according to step B) can, e.g., be induced by a temporary change in the temperature of the contamination-reducing component. For example, a heating coil can be used to temporarily heat up the contamination-reducing component to a temperature of, e.g., above 50° C. Alternatively, a release of adsorbate can be induced by a displacement of the adsorbate from the adsorption sites using a displacement adsorbate of higher adsorption energy. Alternatively in more general terms, a release of sorbate can be induced by a displacement of the sorbate from the sorption sites using a displacement sorbate of higher sorption energy.

The determination of the amount of carbon dioxide can be quantitative or qualitative. In an embodiment, a qualitative determination is performed by comparing the total amount of adsorbate (or generally sorbate) released with the total amount of adsorbate (or generally sorbate) released from a “fresh” contamination-reducing component, i.e. a contamination-reducing component to which—at least approximately—only carbon dioxide is adsorbed (or generally sorbed). Since carbon dioxide is generally more easily released than other adsorbates (or generally sorbates), in particular water, a slight deviation from the value obtained for the “fresh” contamination-reducing component is indicative for a high ratio of the amount of carbon dioxide to the total amount of adsorbate (or generally sorbate), whereas a great deviation is indicative for low ratio of the amount of carbon dioxide to the total amount of adsorbate (or generally sorbate).

Depending on the deviation from the value obtained for the “fresh” contamination-reducing component, the ratio of adsorbed (or generally sorbed) carbon dioxide to the total amount of adsorbate (or generally sorbate) can qualitatively be determined.

As mentioned, the amount of carbon dioxide released can be determined based on the determination of the total amount of adsorbate (or generally sorbate) released. In an embodiment, the process described above can thus comprise between step B) and step C) a further step (“step B′”) of determining the total amount of adsorbate (or generally sorbate) released in step B). This total amount of adsorbate (or generally sorbate) released can be determined, e.g. by measuring the pressure change caused by the release of adsorbate (or generally sorbate). Alternatively, the change in weight of the contamination-reducing component caused by the release of adsorbate (or generally sorbate) can be determined.

According to a further aspect, the present invention further relates to a process for monitoring the sorption capacity, in particular adsorption capacity, of a contamination-reducing component in an electrical apparatus over time, said electrical apparatus comprising a housing enclosing an insulating space and an electrical component arranged in the insulating space, said insulating space comprising an insulation medium which comprises or consists of carbon dioxide. This process comprises the steps of:
α) providing to the insulating space a contamination-reducing component, in particular a moisture-reducing component, more particularly a molecular sieve, with carbon dioxide adsorbed (or generally sorbed) thereto,
β) determining the amount of carbon dioxide in the insulating space over time,
γ) determining from a change measured in step β) the amount of carbon dioxide released from the contamination-reducing component, in particular the moisture-reducing component, more particularly the molecular sieve, over time, and
δ) determining from the amount determined in step γ) the amount of water and/or decomposition products adsorbed (or generally sorbed) by the contamination-reducing component, in particular the moisture-reducing component, more particularly the molecular sieve, and thereby its adsorption capacity (or generally sorption capacity) over time.

Generally, the above defined processes are for determining and/or monitoring the sorption capacity. In this embodiment, the adsorbate is, thus, a sorbate, which is sorbed to the contamination-reducing component or moisture-reducing component, respectively, and in particular to the molecular sieve.

Preferably, any of the above defined processes for determining and/or monitoring the sorption capacity, specifically the adsorption capacity, is carried out after the process for providing the contamination-reducing component to the electrical apparatus defined above.

It is further preferred that in any of the defined processes for determining and/or monitoring the sorption capacity, specifically the adsorption capacity, the electrical apparatus is as defined in above.

If the amount of carbon dioxide increases over time, this is a clear indication that adsorbed (generally sorbed) carbon dioxide is released due to a replacement by water adsorbing (generally sorbing) to the contamination-reducing component and that thus the contamination-reducing component is fully functional.

If on the other hand, carbon dioxide remains stable or even decreases although water is present, this is an indication that the adsorption capacity (generally sorption capacity) is exhausted and that thus the contamination-reducing component needs to be replaced.

In order to allow for a reliable determination in this regard, the process preferably comprises the further step of determining the amount of water in the insulating space over time, in particular in case that the amount of carbon dioxide in the insulating space remains stable or decreases over time.

As mentioned, a molecular sieve, in particular a zeolite, having an average pore size in the range from 5 Å to 13 Å is particularly preferred for its high adsorption capacity towards water and decomposition products, such as hydrogen fluoride. This is illustrated in Table 1 listing the limit capacity, inter alia, of zeolite 5A (having an average pore size of 5 Å) and zeolite 13X (having an average pore size of about 9 Å) towards water and hydrogen fluoride (amongst other compounds). The “limit capacity” means the maximum adsorption capacity of the contamination-reducing component or adsorbent, that is the maximum possible amount of the respective adsorbate (in mole) per weight of the contamination-reducing component or adsorbent (in kilogram), at the temperature of maximum adsorption.

TABLE 1

| | Limit capacity μₗᵢₘ (mol/kg) | | | |
|---|---|---|---|---|
| | Activated | Zeolite | Zeolite | |
| Adsorbent | alumina | 5A | 13X | Sorption type |
| H₂O | 5 | 8 | 13 | physisorption |
| SF₄ | 0.5 | 2.25 | 1 | chemisorption |
| WF₆ | n.a. | 0.15 | n.a. | chemisorption |
| HF | n.a. | 8 | 13 | chemisorption |
| SOF₂ | 0.55 | 3 | 1.3 | chemisorption |
| SF₆ | 0.3 | ~0 | 1.5 | physisorption |

(n.a. = not available)

As shown in Table 1, the limit capacity towards water is higher for the specific molecular sieves mentioned above than for activated alumina. Said molecular sieves also show a high adsorption capacity towards hydrogen fluoride.

The present invention is further illustrated by way of the following example.

### EXAMPLE

According to a specific example of the process of the present invention, a vessel enclosing a volume of 4.6 liter with zeolite 5A is provided. The vessel is then filled with carbon dioxide to a partial pressure of 0.97 bar and the zeolite 5A is allowed to adsorb carbon dioxide, by which adsorption the carbon dioxide partial pressure drops to almost 0.7 bar. About hours after filling the carbon dioxide into the vessel, water is injected. As a result, the carbon dioxide partial pressure rapidly increases to 0.97 bar, i.e. the value prior to initial adsorption. Thus, essentially all carbon dioxide adsorbed during initial adsorption is replaced by water adsorbing to the contamination-reducing component and is thus released.

Throughout this application, the terms “preferable”, “preferred”, “more preferable”, “in particular” shall solely mean “exemplary” and shall therefore signify embodiments or examples only, i.e. are to be understood as optional.

### BRIEF DESCRIPTION OF THE DRAWINGS

The invention is further illustrated by way of the attached

FIG. 1 showing a purely schematic representation of an electrical apparatus according to the present invention, for example a switchgear, and

FIG. 2 showing a presaturation vessel according to embodiments of the present invention.

### DETAILED DESCRIPTION OF THE INVENTION

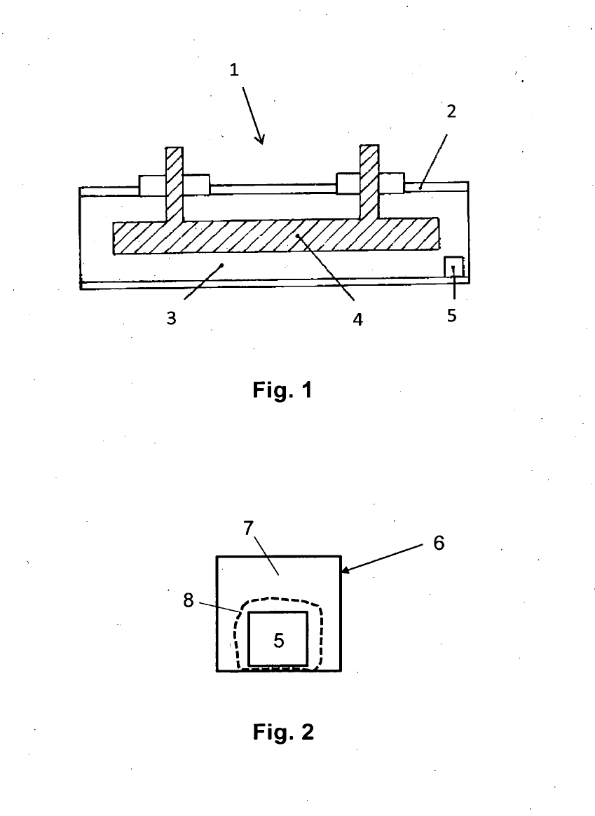

Specifically, the electrical apparatus 1, more particularly the switchgear, shown in FIG. 1 comprises a housing 2 enclosing an insulating space 3 and an electrical component 4 arranged in the insulating space 3. The insulating space 3 contains an insulation medium which comprises or consists of carbon dioxide. In the insulating space 3, a contamination-reducing component 5, more particularly a molecular sieve 5 having an average pore size in a range from 5 Å to 13 Å, is arranged.

FIG. 2 shows schematically a gas-tight closeable and openable presaturation vessel 6 providing a presaturation space 7 for receiving and presaturating with carbon dioxide the contamination-reducing component 5, in particular molecular sieve 5 and preferably zeolite 5. The presaturation vessel 6 may be transferred into the electrical apparatus 1 and may be opened (in particular the component 5 may be removed from the vessel 6) therein in order to expose the contamination-reducing component 5 to the dielectric insulation medium of the electrical apparatus 1. As another embodiment shown in dashed lines, a container 8 or bag 8 may be present for transferring the presaturated contamination-reducing component 5 to the insulating space 3 of the electrical apparatus 1 and to bring it into gas-exchange contact with the dielectric insulation medium of the electrical apparatus 1.

The term “sorption” as used throughout this application is to be interpreted broadly and encompasses any physical or chemical process by which a first substance, i.e. the sorbate, is attached to a second substance, i.e. the sorbent. In particular, it encompasses any binding, capturing or immobilization of the sorbate, for example by physisorption and/or chemisorption.

According to specific embodiments of the present invention, the term “sorption” relates to “adsorption”. In this regard, the terms “sorbed”, “sorbate” and “sorbent” relates to the “adsorbed”, “adsorbate” and “adsorbent”, respectively.

Alternatively or additionally, the term “sorption” can also relate to “absorption”, in the context of which the terms “sorbed”, “sorbate” and “sorbent” relates to the “adsorbed”, “adsorbate” and “adsorbent”, respectively.

As mentioned, the term “contamination-reducing component” encompasses any component suitable for reducing or eliminating the presence of contaminants, in particular moisture (i.e. water) and/or decomposition products and/or any other component the presence of which is not desired. According to specific embodiments, the term “contamination-reducing component” relates to a water-reducing component, in particular encompassing a desiccant.

1. A process for providing a contamination-reducing component to an electrical apparatus, said electrical apparatus comprising a housing enclosing an insulating space and an electrical component arranged in the insulating space, said insulating space comprising an insulation medium which comprises or consists of carbon dioxide, wherein the process comprises the steps of:
a) providing a pre-saturation vessel which is closable in a gas-tight manner and which in its closed state encloses a pre-saturation space, the volume of which being smaller than the volume of the insulating space of the electrical apparatus,
b) placing a contamination-reducing component in the pre-saturation space,
c) filling a pre-saturation gas comprising or consisting of carbon dioxide into the pre-saturation space such as to allow the contamination-reducing component placed in the pre-saturation space to adsorb carbon dioxide, and
d) transferring the contamination-reducing component with the adsorbed carbon dioxide to the electrical apparatus such that during operation of the electrical apparatus it comes into contact with the insulation medium.

2. The process according to claim 1, wherein in step c) the contamination-reducing component is allowed to adsorb carbon dioxide and in step d) the contamination-reducing component with the adsorbed carbon dioxide is transferred to the electrical apparatus.

3. The process according to claim 1, wherein in step d) the contamination-reducing component is transferred into the insulating space of the electrical apparatus.

4. The process according to claim 1, wherein prior or during step d), the contamination-reducing component with the carbon dioxide adsorbed is taken out of the pre-saturation space.

5. The process according to claim 1, wherein the contamination-reducing component is packaged into a container, in particular a bag, prior to being taken out of the pre-saturation space, said container being moveable with regard to the pre-saturation vessel and the electrical apparatus.

6. The process according to claim 5, wherein the container is closeable in a gas-tight manner.

7. The process according to claim 1, wherein in step d) the pre-saturation vessel together with the contamination-reducing component placed in the pre-saturation space is transferred to the electrical apparatus, and after step d) the pre-saturation vessel is opened.

8. The process according to claim 1, wherein the contamination-reducing component is a molecular sieve.

9. The process according to claim 8, wherein the molecular sieve is a zeolite.

10. The process according to claim 8, wherein the molecular sieve has an average pore size greater than 2 Å, preferably greater than 4 Å, more preferably greater than 5 Å, even more preferably greater than 6 Å, and most preferably greater than 8 Å.

11. The process according to claim 8, wherein the molecular sieve has an average pore size from 3 Å to 13 Å, preferably from 5 Å to 13 Å, more preferably from 6 Å to 13 Å or from 6 Å to 12 Å, even more preferably from 7 Å to 11 Å, most preferably from 9 Å to 11 Å.

12. The process according to claim 1, wherein the contamination-reducing component is cooled prior to step d), in particular prior to step c) and/or during step c), and/or is cooled during step d), preferably to a temperature below 10° C., more preferably below 0° C., most preferably below −20° C.

13. The process according to claim 12, wherein the contamination-reducing component is cooled to a temperature which is equal to or lower than 5° C. above the minimum operating temperature of the electrical apparatus, in particular wherein the contamination-reducing component is cooled to a temperature which is equal to or lower than the minimum operating temperature of the electrical apparatus.

14. The process according to claim 1, wherein the number density of carbon dioxide in the pre-saturation space is higher than the number density of carbon dioxide in air at atmospheric pressure.

15. The process according to claim 1, wherein the number density of carbon dioxide in the pre-saturation space is at least approximately equal to the maximum expected number density of carbon dioxide in the insulating space of the electrical apparatus.

16. The process according to claim 1, wherein the partial pressure of carbon dioxide in the pre-saturation space at room temperature is higher than 1 bar, preferably higher than 3 bar, more preferably higher than 5 bar, and most preferably higher than 7 bar.

17. The process according to claim 8, wherein the volume of the pre-saturation space is slightly greater than the volume of the molecular sieves.

18. The process according to claim 1, wherein the insulation medium and the pre-saturation gas have at least approximately the same composition.

19. The process according to claim 1, wherein the insulation medium comprises, apart from carbon dioxide, an additional background gas, in particular selected from the group consisting of: air, air component, nitrogen, oxygen, nitrogen oxides, and mixtures thereof.

20. The process according to claim 19, wherein the ratio of the amount of carbon dioxide to the amount of oxygen ranges from 50:50 to 100:1, preferably from 80:20 to 95:5, more preferably from 85:15 to 92:8, even more preferably from 87:13 to less than 90:10, and most preferably is about 89:11.

21. The process according to claim 1, wherein the insulation medium further comprises an organo-fluorine compound, preferably an organofluorine compound selected from the group consisting of: fluoroethers, in particular hydrofluoromonoethers, fluoroketones, in particular perfluoroketones, and fluoroolefins, in particular hydrofluoroolefins, and mixtures thereof.

22. An Electrical apparatus, comprising a housing enclosing an insulating space and an electrical component arranged in the insulating space, said insulating space containing an insulation medium which comprises or consists of carbon dioxide, wherein in the insulating space a molecular sieve having an average pore size in a range from 5 Å to 13 Å is arranged, wherein the molecular sieve is arranged in the pre-saturation space of a pre-saturation vessel, said pre-saturation vessel being in its opened state.

23. The electrical apparatus according to claim 22, the molecular sieve being a water-reducing component.

24. The electrical apparatus according to claim 22, wherein the molecular sieve has an average pore size greater than 5 Å, preferably greater than 6 Å, and most preferably greater than 8 Å.

25. The electrical apparatuses according to claim 22, wherein the molecular sieve has an average pore size from 6 Å to 12 Å, preferably from 7 Å to 11 Å, more preferably from 9 Å to 11 Å.

26. The electrical apparatus according to claim 22, wherein the insulation medium comprises, apart from carbon dioxide, an additional background gas, in particular selected from the group consisting of: air, air component, nitrogen, oxygen, nitrogen oxides, and mixtures thereof.

27. The electrical apparatus according to claim 22, wherein the ratio of the amount of carbon dioxide to the amount of oxygen ranges from 50:50 to 100:1, preferably from 80:20 to 95:5, more preferably from 85:15 to 92:8, even more preferably from 87:13 to less than 90:10, and most preferably is about 89:11.

28. The electrical apparatus according to claim 22, wherein the insulation medium further comprises an organofluorine compound.

29. The electrical apparatus according to claim 22, wherein the insulation medium further comprises an organofluorine compound selected from the group consisting of: fluoroethers, in particular hydrofluoromonoethers, fluoroketones, in particular perfluoroketones, and fluoroolefins, in particular hydrofluoroolefins, and mixtures thereof.

30. The electrical apparatus according to claim 22, wherein the electrical component is a high voltage unit or a medium voltage unit.

31. The electrical apparatus according to claim 22, wherein the electrical apparatus is a switchgear, in particular a gas-insulated switchgear, or is a part and/or component thereof, in particular a busbar, a bushing, a cable, a gas-insulated cable, a cable joint, a gas-insulated line, a transformer, a current transformer, a voltage transformer, a surge arrester, an earthing switch, a disconnector, a combined disconnector and earthing switch, a load-break switch, a circuit breaker, a convertor building and/or any type of gas-insulated switch.

32. The process for determining the adsorption capacity of a contamination-reducing component in an electrical apparatus, said electrical apparatuses comprising a housing enclosing an insulating space and an electrical component arranged in the insulating space, said insulating space comprising an insulation medium which comprises or essentially consists of carbon dioxide, wherein said process comprises the steps of:
A) providing to the insulating space a contamination-reducing component, with at least one kind of adsorbate adsorbed thereto, said at least one kind of adsorbate comprising carbon dioxide,
B) inducing an at least partial release of adsorbate, from the contamination-reducing component,
B′) determining the total amount of adsorbate released in step B),
C) determining the amount of carbon dioxide released from the contamination-reducing component based on the determination of the total amount of adsorbate released, and
D) determining from the amount determined in step C) the amount of the remaining adsorbates in the contamination-reducing component and thus the adsorption capacity of the contamination-reducing component, with the remaining adsorbates in the contamination-reducing component being water and/or decomposition products,
wherein the release according to step B) is induced by a temporary change in the temperature of the contamination-reducing component, or alternatively the release according to step B) is induced by a displacement of the adsorbate from the adsorption sites using a displacement adsorbate of higher adsorption energy.

33. The process of claim 32, wherein a heating coil is used to temporarily heat up the contamination-reducing component, in particular to a temperature of above 50° C.

34. The process of claim 32, wherein a qualitative determination of the amount of carbon dioxide is performed by comparing the total amount of adsorbate released with the total amount of adsorbate released from a fresh contamination-reducing component, to which at least approximately only carbon dioxide is adsorbed, wherein carbon dioxide is more easily released than the other adsorbates, and a slight deviation from the value obtained for the fresh contamination-reducing component is indicative for a high ratio of the amount of carbon dioxide to the total amount of adsorbate, whereas a great deviation is indicative for a low ratio of the amount of carbon dioxide to the total amount of adsorbate.

35. The process of claim 32, wherein the total amount of the sorbate released is determined by measuring a pressure change caused by the release of the adsorbate.

36. The process of claim 32, wherein the total amount of the sorbate released is determined by determining a change in weight of the contamination-reducing component caused by the release of the adsorbate.

37. The process of claim 32, wherein the contamination-reducing component is a molecular sieve.

38. A process for monitoring the adsorption capacity of a contamination-reducing component in an electrical apparatuses over time, wherein the process comprises the steps of:
α) providing to the insulating space a contamination-reducing component, with at least one kind of adsorbate adsorbed thereto, said at least one kind of adsorbate comprising carbon dioxide,
β) determining the amount of carbon dioxide in the insulating space over time,
γ) determining from a change measured in step β) the amount of carbon dioxide released from the contamination-reducing component, over time, and
δ) determining from the amount determined in step γ) the amount of water and/or decomposition products adsorbed by the contamination-reducing component, and thereby its adsorption capacity over time,
wherein the process comprises the further step of determining the amount of water in the insulating space over time.

39. The process of claim 38, wherein the contamination-reducing component is a moisture-reducing component, in particular a molecular sieve.

40. The process of claim 38, wherein the process comprises the further step of determining the amount of water in the insulating space over time in case that the amount of carbon dioxide in the insulating space remains stable or decreases over time.

41. A process of providing a contamination-reducing component to the electrical apparatus comprising the steps of:
a) providing a pre-saturation vessel which is closable in a gas-tight manner and which in its closed state encloses a pre-saturation space, the volume of which being smaller than the volume of the insulating space of the electrical apparatus,
b) placing a contamination-reducing component in the pre-saturation space,
c) filling a pre-saturation gas comprising or consisting of carbon dioxide into the pre-saturation space such as to allow the contamination-reducing component placed in the pre-saturation space to adsorb carbon dioxide,
d) transferring the contamination-reducing component with the adsorbed carbon dioxide to the electrical apparatus such that during operation of the electrical apparatus it comes into contact with the insulation medium;
e) providing to the insulating space a contamination-reducing component, with at least one kind of adsorbate adsorbed thereto, said at least one kind of adsorbate comprising carbon dioxide,
f) inducing an at least partial release of adsorbate, from the contamination-reducing component,
g) determining the total amount of adsorbate released in step f),
h) determining the amount of carbon dioxide released from the contamination-reducing component based on the determination of the total amount of adsorbate released, and
i) determining from the amount determined in step h) the amount of the remaining adsorbates in the contamination-reducing component and thus the adsorption capacity of the contamination-reducing component, with the remaining adsorbates in the contamination-reducing component being water and/or decomposition products,
wherein the release according to step f) is induced by a temporary change in the temperature of the contamination-reducing component, or alternatively the release according to step f) is induced by a displacement of the adsorbate from the adsorption sites using a displacement adsorbate of higher adsorption energy.

42. The process according to claim 41, including a housing enclosing an insulating space and an electrical component arranged in the insulating space, said insulating space containing an insulation medium which comprises or consists of carbon dioxide, wherein in the insulating space a molecular sieve having an average pore size in a range from 5 Å to 13 Å is arranged, wherein the molecular sieve is arranged in the pre-saturation space of a pre-saturation vessel, said pre-saturation vessel being in its opened state.

43. The process according to claim 1, wherein adsorption relates alternatively or additionally to absorption.
